(19) United States
(12) Patent Application Publication
(10) Pub. No.: US 2006/0276442 A1
(43) Pub. Date: Dec. 7, 2006

(54) METHODS OF FEMALE SEXUAL ENHANCEMENT

(76) Inventor: John R. Woodward, Dallas, TX (US)

Correspondence Address: MICHAEL A. O'NEIL, P.C., 5949 SHERRY LANE, SUITE 820, DALLAS, TX, 75225, US

(21) Appl. No.: 11/463,140

(22) Filed: Aug. 8, 2006

Related U.S. Application Data
(63) Continuation-in-part of application No. 11/459,558, filed on Jul. 24, 2006.
Application No. 11/459,558 is a continuation-in-part of application No. 11/387,260, filed on Mar. 23, 2006.
Application No. 11/387,260 is a continuation-in-part of application No. 11/153,174, filed on Jun. 15, 2005.
Application No. 11/153,174 is a continuation-in-part of application No. 10/936,965, filed on Sep. 8, 2004.

(51) Int. Cl. A61K 31/57

(52) U.S. Cl. 514/177

(57) ABSTRACT

A method of sexual enhancement in women includes the steps of identifying a woman requesting sexual enhancement, assuring that the woman's blood includes estradiol within a first predetermined range and testosterone within a second predetermined range, and thereafter administrating a drug selected from the group consisting of vardenafil hydrochloride and tadalafil prior to sexual activity. The selected drug may be loaded into a starch strip which is then applied to the woman's tongue. Sexual enhancement in women can also be achieved by transdermal or subcutaneous application of the steroid hormone DHEA.

### CROSS-REFERENCE TO RELATED APPLICATIONS

This application is a continuation-in-part application of application Ser. No. 11/459,558 filed Jul. 24, 2006, currently pending, the entire contents of which are incorporated herein by reference; which is a continuation-in-part application of application Ser. No. 11/387,260 filed Mar. 23, 2006, currently pending, the entire contents of which are incorporated herein by reference; which is a continuation-in-part application of application Ser. No. 11/153,174 filed Jun. 15, 2005, currently pending, the entire contents of which are incorporated herein by reference; which is a continuation-in-part of application Ser. No. 10/936,965 filed Sep. 8, 2004, currently pending, the entire contents of which are incorporated herein by reference.

### TECHNICAL FIELD

This invention relates generally to enhancement of sexual arousal and satisfaction in females, and more particularly to the use of drugs already approved for the treatment of erectile dysfunction in males and to the use of the steroid hormone DHEA to stimulate sexual arousal and satisfaction in females.

### BACKGROUND AND SUMMARY OF THE INVENTION

As is universally known, various drugs are now available for the treatment of erectile dysfunction. By stimulating the erectile process, these drugs promote sexual arousal and ultimately sexual satisfaction in men. The first drug of this type to gain widespread acceptance was VIAGRA® (sildenafil citrate) which was introduced in 1998. LEVITRA® (vardenafil hydrochloride) and CIALIS® (tadalafil) have been introduced more recently. Despite the fact that drug therapy which provides sexual enhancement in males has been available for at least fifteen years, corresponding sexual enhancement for women has not heretofore been available.

The present invention comprises methods of female sexual enhancement which overcomes the foregoing and other problems which have long since characterized the prior art. In accordance with the broader aspects of a first embodiment of the invention, the same types of drugs that are utilized to treat erectile dysfunction in men are used to provide sexual enhancement in women. More particularly, it has been demonstrated with a drug selected from the group consisting of vardenafil hydrochloride and tadalafil prior to sexual activity results in significant enhancement of sexual arousal and sexual satisfaction in females.

More particularly, the initial step in the process comprising the first embodiment of the invention comprises identifying a woman desiring sexual enhancement. The process can be started either at the request of the woman herself, or at the suggestion of her physician. Thereafter, the woman's blood is tested to determine the levels of estradiol and testosterone. If the woman's blood contains an estradiol level within a first predetermined range and a testosterone level within a second predetermined range, the process of the present invention continues. If not, the woman is treated utilizing conventional techniques to bring the level of estradiol in her blood to within the first predetermined range and/or to bring the level of testosterone in her blood to within the second predetermined range.

After the required amount of estradiol and the required amount of testosterone in the woman's blood has been assured, the woman is treated with a drug selected from the group consisting of vardenafil hydrochloride and tadalafil. In most instances the drug is self-administered by the woman desiring sexual enhancement. The typical dosage is ½ of the recommended dosage of the same drug for the treatment of erectile dysfunction in males. The selected drug is preferably administered at least 15-20 minutes before sexual activity.

The use of the drug VIAGRA® (sildenafil citrate) to achieve sexual enhancement in women has heretofore been attempted. The use of VIAGRA® (sildenafil citrate) for such purposes has been found to be unsatisfactory because of unacceptable side affects. The unacceptable side effects that are observed in women using VIAGRA® (sildenafil citrate) for sexual enhancement include:
Headaches
Facial flushing
Nasal congestion
Indigestion
Bluish tinge to vision lasting up to a few hours
Blindness.
   Due to the foregoing side affects, it has been determined that VIAGRA® (sildenafil citrate) cannot be safely utilized for sexual enhancement in women.

In accordance with a second embodiment of the invention a drug selected from the group consisting of LEVITRA® (vardenafil hydrochloride) and CIALIS® (tadalafil)is loaded into an appropriately flavored starch strip. The starch strip is then applied to the tongue of a woman having blood levels of estradiol and testosterone within the normal ranges. The time period between application of the drug-loaded strip to the patient's tongue to full effect of the selected drug is typically about 15 to 20 minutes.

In accordance with a third embodiment of the invention the steroid hormone DHEA is administered either transdermally or subcutaneously to women having blood levels of estradiol and testosterone within normal ranges. The result is enhanced female sexual pleasure and satisfaction including sexual arousal and achievement of orgasm.

### BRIEF DESCRIPTION OF THE DRAWING

A more complete understanding of the present invention may be had by reference to the following Detailed Description when taken in connection with the accompanying Drawing, wherein:

FIG. 1 is a flowchart illustrating a method comprising a second embodiment of the present invention; and

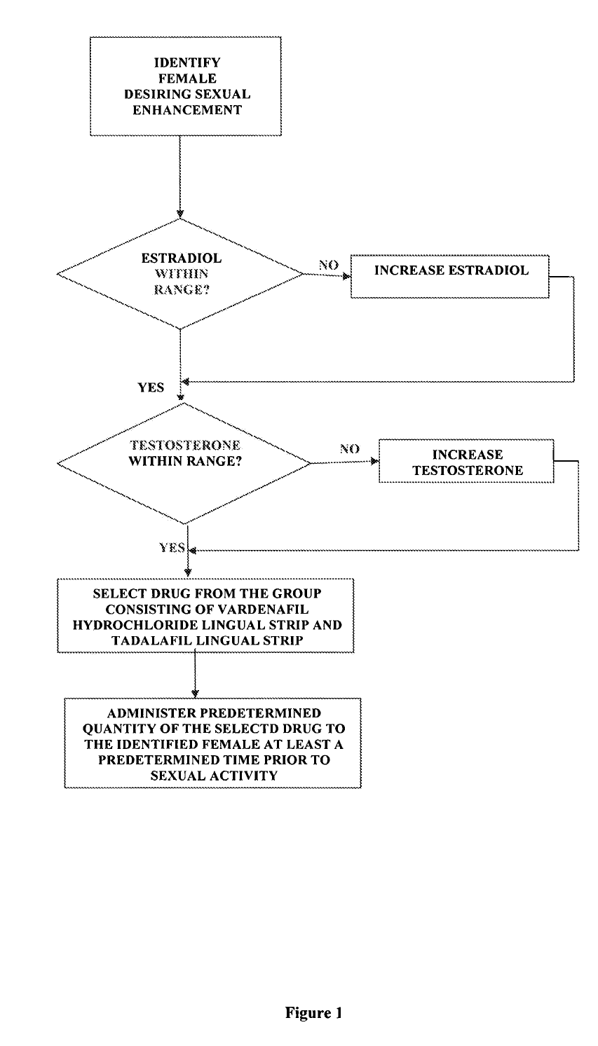

FIG. 2 is a flowchart illustrating a method comprising a third embodiment of the present invention.

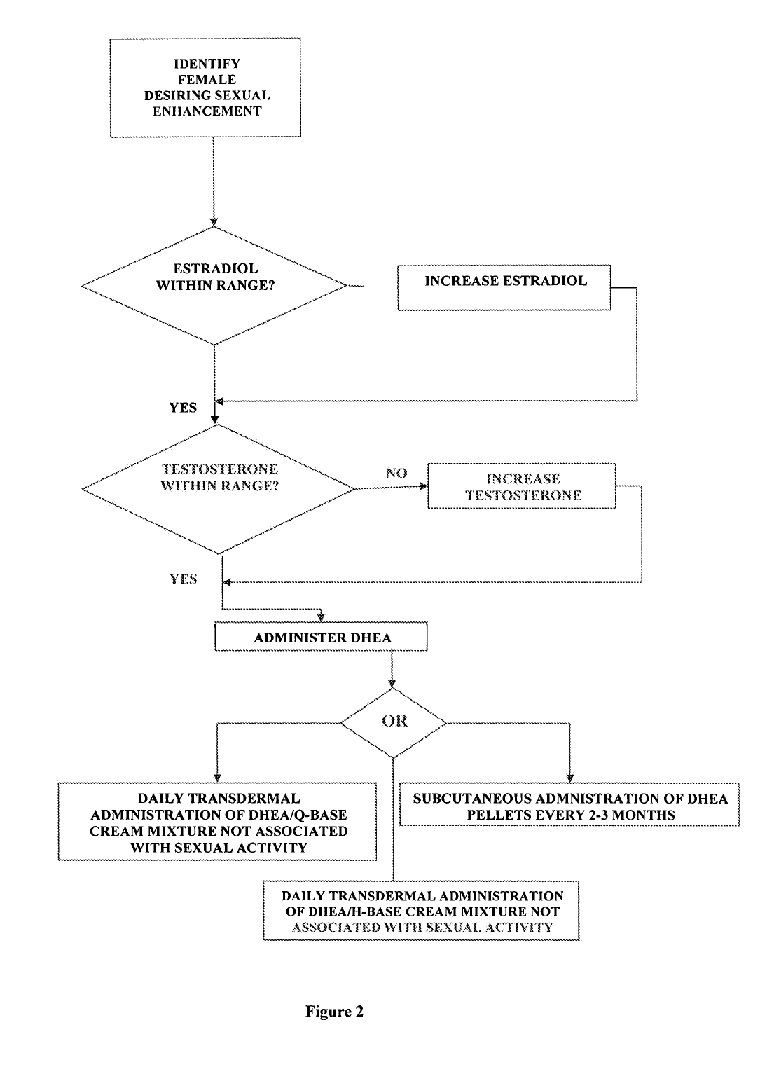

### DETAILED DESCRIPTION

The first embodiment of the invention comprises a method of female sexual enhancement. The first step in the practice of the invention comprises the identification of a woman desiring sexual enhancement. Typically this step occurs as part of a consultation between the woman and her physician. The consultation may be specially scheduled in order that the woman may avail herself of the present invention. More often, however, the identification step occurs during a meeting of the woman with her physician for other purposes, such as a regularly scheduled consultation, an examination unrelated to the present invention, a procedure, etc. The woman may be identified as a participant in the method of the present invention either at her own request or at the suggestion of her physician.

After her identification as a participant in the method of the present invention, the woman's blood is tested for the levels of estradiol and testosterone therein. The successful practice of the method of the present invention requires that the woman's blood contains an estradiol level within a first predetermined range and a testosterone level within a second predetermined range. For example, one laboratory has established the first predetermined range as between about 50 picograms and about 150 picograms of estradiol per 1 cc of blood serum, and has established the second predetermined range as between about 200 picograms and about 540 picograms of testosterone per 1 cc of blood serum. Other laboratories have similar, but not necessarily identical, definitions of the first and second predetermined ranges.

If the blood test reveals that the amount of estradiol in the woman's blood is below the first predetermined range, the amount of estradiol in the woman's blood is raised utilizing conventional practices and procedures. For example, non-oral, bio-identical estradiol is administered as a transdermal cream, as a patch, or as a subcutaneous pellet. Similarly, if the blood test reveals that the amount of testosterone in the woman's blood is below the second predetermined range, conventional practices and procedures are undertaken for the purpose of raising the amount of testosterone in the woman's blood. For example, non-oral, bio-identical testosterone is administered as a transdermal cream, as a patch, or as a subcutaneous pellet. In some instances it may be necessary to raise both the amount of estradiol and the amount of testosterone in the woman's blood.

After the required levels of estradiol and testosterone in the woman's blood have either been confirmed or established, a drug selected from the group consisting of vardenafil hydrochloride and tadalafil is administered to the woman prior to sexual activity. In most instances the selected drug is self-administered. It has been determined that the selected drug should be administered at least 15-20 minutes prior to the beginning of sexual activity. The optimum time interval between administration of the selected drug and the beginning of sexual activity may vary depending on the particular circumstances and is best determined by the woman through experimentation. It has also been determined that the appropriate dosage of a drug selected from the group consisting of vardenafil hydrochloride and tadalafil in order to enhanced sexual stimulation and satisfaction is one half of the dosage of the same drug that is recommended for administration to males to treat erectile dysfunction. The exact amount of the drug that is appropriate for a particular woman may vary depending upon a variety of circumstances all of which are best explored through consultation between the woman and her physician.

It has been determined that the method of the present invention results in substantial sexual enhancement in women, including in particular substantially improved sexual arousal and substantially improved sexual satisfaction. This in turn results in an improved overall feeling of wellness, an improved sexual relationship between the woman and her partner, and an overall improvement in self esteem.

### CLINICAL STUDY PROTOCOL

Enhancing the Efficacy of Vardenafil Hydrochloride

Summary:

The ability of vardenafil hydrochloride to stimulate female sexual response depends upon the circulating levels of sex steroids. This hypothesis has been tested in a double-blind, placebo-controlled study with measurement and adjustment of estradiol and testosterone at UCLA Medical Center in Los Angeles, Calif.

Introduction:

Previous studies by Glaxo-Smith Kline indicated that vardenafil hydrochloride was ineffective in women. This occurred because normal estradiol and testosterone levels were not established or achieved with non-oral hormone replacement before the drug was used. Experiences over the past 2 years indicate that vardenafil hydrochloride is even more effective (lower dosage) in women than in men when used correctly.

Objectives:

The purpose of the present study is to determine the efficacy of vardenafil hydrochloride in stimulating female sexual response in women with adequate levels of estradiol and testosterone.

Study Design:

The study group consisted of 100 healthy, sexually active women between ages 20 and 70. The women were not taking exogenous hormones, either oral contraceptives or postmenopausal hormone therapy. The participants were cautioned to use appropriate contraception, as the safety of vardenafil hydrochloride during pregnancy has not been definitively demonstrated.

After a 4-week baseline period, the participants were randomly assigned by a computer-generated schedule to receive either vardenafil hydrochloride in doses of ½ of a 20 mg tablet at least 1 hour prior to each sexual experience and no later than 8 hours, or a placebo for a period of 2 months, followed by a 2-month period with randomized cross-over. Serum estradiol and testosterone levels were measured at baseline by Interscience Institute, Inglewood, Calif. Blood samples were obtained approximately one week before menses. A normal level of estradiol, between about 50 pg/ml and about 150 pg/ml, and a normal level of testosterone, between about 200 pg/ml and about 540 pg/ml, were required for inclusion in the study.

Sexual response and any side effects were recorded monthly using one of the available and accepted methods for this purpose, such as the Sexual Function Index. In addition, each subject kept a daily event log diary.

A second study is recommended for women with low blood levels of estradiol and testosterone, with randomization to either placebo or treatment with supplemental non-oral estradiol or testosterone administered by transcutaneous cream, cutaneous patch or subcutaneous pellets.

Inclusion Criteria:

A) Sexually active (defined as coitus, foreplay, manual or oral stimulation).

B) Satisfying monogamous relationship with a partner.

C) No evidence of sexual dysfunction in partner.

D) Distress over reduced level of sexual desire, arousal, and/or low libido during sexual intercourse or masturbation, and difficulty achieving orgasm.

E) Healthy by medical history and physical examination.

F) BMI between 20 and 30.

G) Voluntary consent to participate following full explanation of nature and purpose of study by signing an IRB-approved protocol.

Exclusion Criteria:

A) Use of exogenous hormones.

B) History of vaginismus or vulvodynia dyspareunia.

C) Evidence of clinical depression.

D) Use of drugs that induce sexual dysfunction (such as SSRIs, GnRH agonist, adrenoreceptor antagonist) within 3 months of enrollment.

Ethical Aspects:

The method of the present invention provides means for women to achieve maximum sexual satisfaction.

A second embodiment of the invention comprises an improved method of administering vardenafil hydrochloride and tadalafil to women. The method is intended for use by sexually mature women who have blood levels of estradiol and testosterone within the normal range and are using effective contraception.

The first step of the method comprises providing an appropriately flavored starch strip. Typical flavors include peppermint, honey, raspberry, lemon, and other well-known and widely used flavors. Regardless of the flavor selection, the strip is loaded with either 10 mg. of vardenafil hydrochloride or 10 mg. of tadalafil.

The selected drug is administered by placing a flavored strip having the selected drug loaded therein on the female patient's tongue. The delivery system of the present invention causes the drug to enter the patient's bloodstream substantially immediately. The time period between the application of the drug-loaded strip to the patient's tongue to full effect of the selected drug is typically about 15 to 20 minutes. The selected drug is thereafter effective in providing enhanced sexual stimulation in female patients for eight hours or more.

Both vardenafil hydrochloride and tadalafil have been widely used for years and both have exhibited remarkable safety profiles.

A third embodiment of the present invention comprises a new use of a well-established steroid hormone. The invention includes both the topical application of the hormone in cream form and the subcutaneous implantation of the hormone in pellet form for the purpose of female sexual enhancement.

The hormone is dehydroepiandrosterone (DHEA) and the carrying agent for its topical use is a proprietary compound, Q-base cream. Q-base cream is available to compound pharmacies all over the U.S. and consists of the following formulation:
purified water, cyclomethicone, octyl stearate, arlacel 165 (PEG-100 stearate/glyceryl stearate), sorbitol, green tea extract (organic), stearyl alcohol, cyclopentasiloxane and PEG/PPG-18/18 dimethicone, gingko extract (organic), ginseng extract (organic), tocotrienols, vitamin E acetate, wheat germ oil, edetate disodium, xanthan gum, vitamin A palmitate, coenzyme Q10, methylchloroisothiazolinone/methylisothiazolin one.

Alternatively, the carrying agent for the DHEA may comprise H-base cream. The formula for H-base cream is as follows:
water, glycerin, canola oil, stearic acid, cetyl alcohol, PEG-100 stearate, glyceryl stearate, dimethicone, magnesium aluminum silicate, propylene glycol, triethanolamine, polysorbate 60, xanthan gum, bitter almond kernel oil, aloe vera, grape seed extract, wheat germ oil, vitamin E acetate, vitamin A palmitate, Vitamin C palmitate, tetrasodium EDTA, potassium sorbate, diazolidinyl urea. H base cream is a proprietary product produced by Professional Compounds Centers of America and licensed by it.

The method of application of either the Q-base cream/DHEA mixture or the H-base cream/DHEA mixture in females is local application to the erectile tissue of the nipples and glans clitoris. The ratio of ingredients comprising the Q-base cream/DHEA mixture is between about 25 mg and about 50 mg of DHEA to about 1 cc of Q-base cream. The ratio of ingredients comprising the H-base cream/DHEA mixture is substantially the same. The resulting mixture is applied one time per day but not related to intercourse. Implantation of subcutaneous DHEA pellets is at the strength of about 25 mg of DHEA per pellet at intervals of between about 2 months and about 3 months. Identical blood levels are achieved with both methods of application. Normal levels are considered to be 50-100 picograms per milliliter.

The method comprising the third embodiment of the invention enhances female sexual pleasure and satisfaction including sexual arousal and achievement of orgasm. Both quality and quantity of orgasmic response are enhanced as well as duration of sexual pleasure. In addition to the benefits derived from sexual enhancements, the use of DHEA cream or pellets has been shown to provide some or all of the following benefits: weight loss; increase in muscle mass; improved muscle to fat ratio; general enhancement of well-being; possible extension of life span; mood elevation; increased energy and stamina (or decreased fatigue); improved lipid profile; prevention of damage to blood vessel walls; prevention or treatment of osteoporosis; treatment of depression; and prevention and treatment of insomnia and headache.

Many illnesses thought to be psychosomatic are in fact precipitated by a state of DHEA deficiency. Low levels of DHEA may be found in connection with cerebral vascular accidents; hypercholesterolemia; arthritis; diabetes; cancer; Alzheimer's disease; and idiopathic autoimmune diseases such as multiple sclerosis, lupus, and fibromyalgia. DHEA levels peak at age 25 and gradually decrease for the remainder of life in both males and females. DHEA is produced by the zona reticularis of the adrenal. Humans produce DHEA in greater quantity than any other species. Even non-human primates have about 10% of the relative serum level of DHEA seen in humans. The fact that rodents produce so little DHEA makes the results of experiments conducted with these laboratory animals very controversial.

The safety profile of DHEA administration has been extraordinary. The only complications in women relate to excess androgen production if blood levels of DHEA exceed 100 picograms per milliliter. Complications include, but are not limited to, male pattern hair loss, excess facial hair growth, and acne.

In an unpublished clinical study of the third embodiment of the invention over the past three years patient acceptance and satisfaction has been clear-cut and in excess of 95%. The response is dependent on the patient having normal blood levels of estradiol and testosterone as well. A normal level of DHEA is about 50 to about 100 picograms per milliliter. A normal level of estradiol is about 50 to about 100 picograms per milliliter and a normal level of testosterone is about 200 to about 540 picograms per milliliter. Normal ranges for all these levels can vary from laboratory to laboratory.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. A method of providing sexual enhancement in women comprising the steps of:
identifying a woman desiring sexual enhancement;
testing the blood of the identified woman to determine the amounts of estradiol and testosterone therein;
assuring that the identified woman's blood includes estradiol within a first predetermined range of between about 50 pg/ml to about 150 pg/ml;
assuring that the identified woman's blood includes testosterone with a second predetermined range of between about 200 pg/ml to about 540 pg/ml;
thereafter administering to the identified woman a predetermined amount of the steroid hormone DHEA.

2. The method of providing sexual enhancement in women according to claim 1 wherein the steroid hormone DHEA is administered to the identified woman transdermally.

3. The method of providing sexual enhancement in women according to claim 2 further characterized by:
preparing a mixture of Q-base cream and the steroid hormone DHEA at a ratio of between about 25 mg and about 50 mg of DHEA to about 1 cc of Q-base cream; and
transdermally administering the resulting mixture to the identified woman.

4. The method of providing sexual enhancement in women according to claim 3 wherein the Q-base cream/DHEA mixture is applied to the nipples and glans clitoris of the identified woman.

5. The method of providing sexual enhancement in women according to claim 4 wherein the Q-base cream/DHEA mixture is applied once daily.

6. The method of providing sexual enhancement in women according to claim 1 wherein the steroid hormone DHEA is administered subcutaneously.

7. The method of providing sexual enhancement in women according to claim 6 wherein the steroid hormone DHEA is administered by subcutaneous implantation of DHEA pellets.

8. The method of providing sexual enhancement in women according to claim 7 wherein the administration of the steroid hormone DHEA is carried out by implementation of subcutaneous DHEA pellets each comprising 25 mg of DHEA.

9. The method of providing sexual enhancement in women according to claim 8 wherein the step of implanting subcutaneous DHEA pellets is carried out at intervals of between about 2 months and about 3 months.

10. The method of providing sexual enhancement in women according to claim 2 further characterized by:
preparing a mixture of h-base cream and the steroid hormone DHEA at a ratio of between about 25 mg and about 50 mg of DHEA to about 1 cc of h-base cream; and
transdermally administering the resulting mixture to the identified woman.

11. The method of providing sexual enhancement in women according to claim 10 wherein the h-base cream/DHEA mixture is applied to the nipples and glans clitoris of the identified woman.

12. The method of providing sexual enhancement in women according to claim 11 wherein the h-base cream/DHEA mixture is applied once daily.
